(19) United States
(12) Patent Application Publication
(10) Pub. No.: US 2024/0412842 A1
(43) Pub. Date: Dec. 12, 2024

(54) COMPUTER IMPLEMENTED SYSTEM AND METHOD FOR OPTIMIZING AN EXERCISE PROGRAM BASED ON PARTICIPANT'S GOALS

(71) Applicant: LF3 Inc., Ottawa ON (CA)

(72) Inventors: Jenna YORK, Ottawa ON (CA); Michael DONNELLY, Ottawa ON (CA)

(73) Assignee: LF3 Inc., Ottawa ON (CA)

(21) Appl. No.: 18/700,728

(22) PCT Filed: Dec. 29, 2021
(86) PCT No.: PCT/CA2021/051895

§ 371 Date: Apr. 12, 2024

(51) Int. Cl. G16H 20/30; G16H 10/60; G16H 40/67; G16H 50/20

(52) U.S. Cl. CPC G16H 20/30; G16H 10/60; G16H 40/67; G16H 50/20

(57) ABSTRACT

There is provided a computer implemented system and method for optimizing an exercise program based on a participant's goals and criteria.

### FIELD OF THE INVENTION

The present invention relates to the field of personal fitness and wellness and in particular to a computer implemented system and method for optimizing an exercise program based on participant's goals.

### BACKGROUND

Becoming physically fit or maintaining physical fitness is a priority for many people for a multitude of different reasons. Reasons may include for example improving health, changing personal appearance or body composition. While it is a priority for many people, people often have difficulty designing appropriate workout programs for their workout goals and current level of fitness. While personal trainers can provide workout programs and dietary plans to meet an individual's workout goals, the cost of personal training can be prohibitive to many people. In addition, given people's busy schedules and the limited availability of some personal trainers, scheduling personal training can be challenging. Moreover, many people prefer to workout at home on their own schedule.

While there are numerous online fitness programs, many online fitness programs are not personalized to reflect an individual's current level of fitness and their fitness goals. In addition. many online fitness programs do not monitor an individual's fitness and do not allow for changes to a fitness program based on changes to an individual's level of fitness or a change in fitness. Accordingly, there is a need for a system to develop a fitness program that at least partially addresses one or more limitations of the prior art.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a computer implemented system and method for optimizing an exercise program based on user's goals. In accordance with an aspect of the invention, there is provided a computer implemented method for generating a workout or a fitness program comprising a plurality of workouts, wherein each of the workout comprises one or more exercises, the method comprising receiving, by a server computing device, a request for a workout or a fitness program, wherein each request comprises one or more criteria, wherein each of the one or more criteria is selected from one or more fitness goals, workout type and workout length, wherein each request comprises a user submitting a request via a user interface on a user device, the user device communicating the request(s) with the server computing device optionally via the internet; generating, by the server computing device, a workout or fitness program based on the one or more criteria; and displaying the workout or fitness program to the user.

In accordance with another aspect of the invention, there is provided a system for generating a workout having a plurality of exercises, the system comprising one or more exercise databases; and a workout generating engine located on a server computing device operatively connectable to one or more user interfaces, the workout generating engine comprising one or more workout templates stored in a memory; the workout generating engine configured to retrieve one or more exercises from the one or more exercise databases and populate a selected template to generate a workout based on user workout criteria.

In accordance with another aspect of the invention, there is provided a system for generating a fitness program having a plurality of workouts, the system comprising a fitness program generating engine located on a server computing device operatively connectable to one or more user interfaces, the fitness program generating engine comprising one or more fitness program templates stored in a memory; the fitness generating engine configured to retrieve one or more workouts from one or more workout databases or from the system for generating a workout in accordance with the invention and populate a selected template to generate a fitness program based on user criteria.

In accordance with another aspect of the invention, there is provided a computer implemented method for generating a fitness program for a participant comprising a plurality of workouts, wherein each of the workout comprises one or more exercises, the method comprising receiving, by a server computing device, a request for a fitness program, wherein each request comprises one or more criteria, wherein each of the one or more criteria is selected from fitness level of participant, one or more fitness goals of the participant, current activities of the participant, wherein each request comprises a user submitting a request via a user interface on a user device, the user device communicating the request(s) with the server computing device optionally via the internet; generating, by the server computing device, a workout or fitness program based on the one or more criteria; and displaying the workout or fitness program to the user.

The foregoing and other objects, features, aspects and advantages of the present disclosure will become more apparent from the following detailed description, taken in conjunction with the accompanying drawings which description is by way of example only.

### BRIEF DESCRIPTION OF THE FIGURES

These and other features of the invention will become more apparent in the following detailed description in which reference is made to the appended drawings.

FIG. 1 shows a diagram for a computer implemented method for generating a fitness program in accordance with one embodiment of the invention;

FIG. 2 shows a diagram for a computer implemented method for generating a workout in accordance with one embodiment of the invention.

FIGS. 3 to 6 show a series of a series of questions to determine how a client is feeling about their training routine, in accordance with one embodiment of the invention.

FIG. 7 shows a query to determine a client's fitness goals, in accordance with one embodiment of the invention.

FIG. 8 shows a query to determine a client's exercise ability level, in accordance with one embodiment of the invention.

### DETAILED DESCRIPTION

There is provided a computer implemented system and method for optimizing an exercise/fitness program based on the participant's goals and current fitness levels and optionally available time, schedule and available equipment and other factors. In some embodiments, the computer implemented system and method include a diet and/or sleep module.

The system and method are configured to provide an exercise/fitness program that is responsive to changes in the participant's fitness, health, available time, schedule, and available equipment amongst other changeable variables to facilitate the attainment of the participant's goals and optional uses an artificial intelligence optimization module trained with a plurality of training sets generated from a plurality of participants' data. Optionally, the plurality of participants whose data from the training sets represent a plurality of different potential participant types (i.e., populations) including untrained, partially trained, trained, elite athlete. Each population of potential participants can be further categorized based on age, sex, pre-existing conditions amongst other categories that would be known to one skilled in the art.

In some embodiments, modification of the exercise/fitness program is automatically generated based on user's or participant's input. User's or participant's input may be provided manual via an appropriate interface. Participant's input may also be provided by pairing or linking wearable trackers that the participant is wearing to method and system.

The computer implemented system and method is also configured to generate appropriate workouts and optionally diet plans based on the current fitness and health status of the participant. Optionally, the computer implemented system and method utilizes an artificial intelligence optimization module to assess the participant and select and/or modify workouts and diet plans based on closest cohort analysis.

In some embodiments, the computer implemented system and method include a form tracking module that provides video analysis of the participant's form when an exercise is completed. Such form tracking is known in the art and includes artificial intelligence-based analysis of a body's pose and movement by detecting and analyzing position and movement of the body by skeleton, contour and/or volume modeling.

In some embodiments, the computer implemented system and method utilize artificial intelligence to optimize and modify fitness programs based on a participant's profile. Optionally, the system and method collect and track participants' progress by collecting and collating participant data. Optionally, the data is made anonymous. From this data, a plurality of training sets is prepared, and these training sets are used to train the fitness program engine to optimize the program based on participant profile.

The methods of the invention comprise receiving, by a server computing device, a request for a workout or a fitness program, wherein each request comprises one or more criteria, wherein each of the one or more criteria is selected from one or more fitness goals, workout type and workout length, wherein each request comprises a user submitting a request via a user interface on a user device, the user device communicating the request(s) with the server computing device optionally via the internet; generating, by the server computing device, a workout or fitness program based on the one or more criteria; and displaying the workout or fitness program to the user. In some embodiments, the user is a health care professional, fitness professional or the participant.

The system and method include one or more databases stored on an accessible memory. Databases include participant databases that include participant profiles and participant data, exercise databases, nutrition databases, and workout databases. In some embodiments, each entry in a database includes or is operatively associated with appropriate metadata tags that facilitate searching and/or indexing of the database. In some embodiments, one or more databases are linked such that an update of an entry in one database is repeated.

The database comprising a plurality of exercises and optionally exercise/workout programs. The database of exercises and exercise programs includes entries for each exercise that optionally includes body part targeted and/or primary muscles, required equipment, difficulty level, calories burned. Each exercise may further be classified as a cardiovascular exercise, strength exercise, mobility exercise, relaxation exercise or other including but not limited to “weight-lifting”, “interval training”, “resistance training”, “high-intensity interval training” or HIIT, “high intensity” and “cross fit”. Optionally, each entry for an individual exercise may be further defined as being part of a set exercises/fitness program, as being an alternative to a different exercise and/or complementary to a different exercise and/or appropriate for a specific type of sport and/or appropriate for specific type of rehabilitation and/or any counterindications, for example not appropriate for a user with a specific type of injury or disability. In some embodiments, each entry includes a video or link to a video that shows how to perform the exercise.

Optionally, the one or more exercise databases are configured to populate a training template based on a set of criteria. Criteria includes but is not limited to fitness level of user, availability of equipment, time, workout type, amongst others. Workout type includes but is not limited to cardiovascular, weight training, interval training, recovery, mobility, stretching, etc. In some embodiments, the type of workout can be further defined.

The training templates are stored on memory accessible by the system and methods of the invention. Optionally, there are a plurality of training templates that are optionally organized based on type of workout and/or length of workout. In some embodiments, the templates are searchable. A completed training template forms a workout that is optionally saveable for later retrieval from memory. Workouts are optionally tagged with appropriate metadata and/or titled to facilitate searching and indexing.

In some embodiments, once a workout is created, the system and method store the workout optionally with associated metadata in a database. Optionally, the system and method determine the likely fitness parameters of the workout including estimated caloric expenditures, intensity, predicted impact on physiological parameters including heart rate and aspiration rate. In some embodiments, where users wear a fitness tracker, the system and method are configured to compare predicted impact to actual impact and optionally modify the prediction based on real world data. In still other embodiments, predicted impact is based on an individual's physiological/physical parameters and is optionally adjusted as these parameters change.

In some embodiments, the system and method are configured to identify substantially equivalent workouts or alternative activities based on a definable parameter, for example predicted calorie burn, for example, running, hiking or cycling for specified times may be equated to a specific cardiovascular exercise class that is scheduled in the fitness program. Optionally, the system and method are configured to allow a user to request an alternative activity for a scheduled workout. In such embodiments, the participant may open access the scheduled workout in the interface, optionally scheduled as a meeting in a calendar, and request an alternative. In some embodiments, the system and method provide a prompt into which the user can input the proposed alternative. Optionally, the system and method provide in real time parameters the alternative activity must meet to be a true alternative. If the alternative activity is found to not be a true alternative to the schedule workout, the method and system automatically adjust the fitness program.

The computer implemented system and method further provides for optimization of an exercise/fitness program based on the user's goals and current fitness levels and optionally available time, schedule and available equipment and others.

In some embodiments, the computer implemented system and method are configured to interface with a scheduling/calendar module or system to provide reminders and alerts to participant. Optionally, any changes in the program are translated into updated calendar entries.

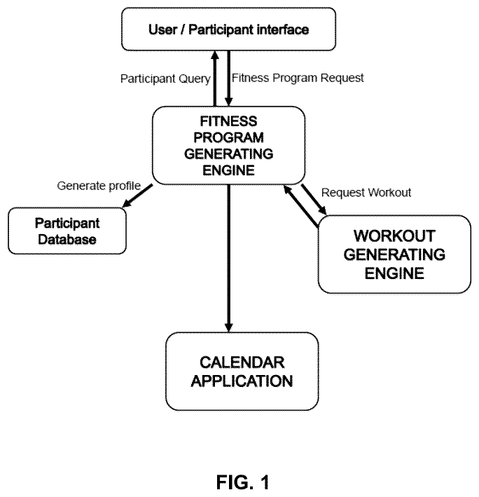

FIG. 1 shows a diagram for a computer implemented method for generating a fitness program.

The method can be completed by the individual participating in the fitness/health program (“participant”), by a health care professional or a fitness professional. Particularly, the method provides a computer implemented method for generating a fitness program automatically from input regarding the participant. Input regarding the participant includes fitness goals, current fitness level and health status including physiological data, available time to commit, schedule, availability of equipment amongst other factors that may impact an individual's ability or desire to complete or continue a fitness/health program. In some embodiments, the method may receive third-party health and fitness data including data from wearable fitness or health devices.

Physiological data includes age, sex, height, body weight, body fat, body composition, heart rate, aspiration rate, blood oxygenation, blood glucose, hydration, basal metabolic rate, caloric expenditure, and VO_{2 max}. Baseline physiological data can be measured by means known in the art. In some embodiments, the physiological data comprises bioimpedance analysis data and 3-D body scan data. Mobility data with respect to the participant may also be inputted. Non-limiting examples of these mobility data can include transfer skills, gait, and the like. Various measurements including hip, waist, thigh, arm, neck may also be provided.

Non-limiting examples of methods that can be used to perform these measurements can include Bio-electrical Impedance Analysis (BIA) or a three-dimensional body scan (3D body scan). Non-limiting examples of devices that can be used by methods that can be used to perform these measurements can include an Oshenwatch, FitBit Aria Air smart scale, FitBit Dara smart body BMI scale, FitBit VersaPolar Heart Rate Monitor, Garmin HRM-Pro, Perifit pelvic floor trainer, Garmin HRM-Pro Premium heart rate strap, Body fat calliper, O2 activity tracker, Apple Watch, and the like.

Optionally, these physiological data, physical aspect data and mobility data can be used to establish what can be known to a person skilled in the art as baseline profile. The baseline profile can be compared to any combination of physical aspect measurements and/or mobility measurements obtained in the future to evaluate the progress of the participant. In some embodiments, a series of exercises and/or physical tasks is included to assess baseline fitness. Appropriate sets of exercises and/or physical tasks useful in assessing baseline fitness are known in the art.

In some embodiments, the method schedules and/or requests that a baseline fitness test be completed to track changes in fitness.

Input may be provided via an interface, on-line portal, computer or “smart” device application or other means known in the art. Appropriate interfaces are well known in the art and include a graphical user interface (GUI) on a computing device screen including “smart” devices and/or touch screen devices including tablets.

The method initiates with a step of receiving input via the interface regarding the individual completing the fitness program that form one or more criteria that the fitness program must meet. Criteria include one or more fitness/health goals, workout type and workout length/frequency.

Optionally, there may be one or more pre-initiation steps. Pre-initiation steps may include registering a user and/or an individual completing the fitness program (“participant”) and/or logging into the method. A profile of the participant is created and optionally stored in an appropriate database.

Input of relevant information into a participant's profile is completed by presenting a series of questions to the individual inputting the information. The series of questions is designed to determine fitness/health goals, current health and fitness level, identify barriers to completing the program including physical, mental, equipment and scheduling and/or time constraints. Optionally, health and fitness data including third-party health and fitness data is imported into the participant's profile. In some embodiments, the method is configured to allow for sinking or sharing of information with authorized third-party applications and systems known in the art.

Using the inputted information, a baseline fitness/health profile of the participant is generated. Optionally, the baseline fitness/health profile of the participant maybe updated at periodic intervals. In some embodiments, the system and method are configured to alert when an update baseline is required. This ensures that the fitness/health program continue to meet the participant's needs and goals.

From the inputted information a request for a fitness program is generated and received by a server computing device of the system for generating a fitness program. The fitness program generating engine receives and processes the request.

In one embodiment, the fitness program generating engine based on the criteria in the requests selects an appropriate fitness program template. In alternative embodiments, the fitness program generating engine generates an appropriate fitness program template from the plurality of fitness program templates stored in system memory.

Each fitness program template defines a period of time, appropriate periods of time include one week, one month, 3 months, 6 months or one year. The period of time is optionally based on the fitness goals of the participant and the length of time it generally takes to see a result based on that goal and in view of the current fitness level of the participant. For example, improvement in cardiovascular health as measured by reduction in resting heart rate, reduction in blood pressure and improved aerobic fitness as measured by VO2MAX may be visible with a sustained program of 6 to 8 weeks, improved muscular fitness including increased strength may be visible in 3 to 6 months.

Each template defining a period of time is broken into defined segments, for example each segment may be 1 week in length. Within each segment, subsegments are further defined, for example days. Individual subsegments are defined as an active subsegment where a specific workout or workout substitute is scheduled or a rest subsegment. Based on the criteria, the fitness program engine may further define an active subsegment based on workout type.

The fitness program engine based on the criteria will retrieve one or more workouts from one or more workout databases or request an appropriate workout from the workout generating engine and populate the selected template to generate a fitness program based on the participant's criteria. The fitness program optionally includes group workouts, on-line workouts, workouts with a personal trainer and individual exercise.

In some embodiments, the fitness program engine includes a workout replacement module that allows a participant to substitute a scheduled workout with an alternative activity. In some embodiments, the workout replacement module compares the alternative activity with the scheduled activity and determine if the two are equivalent. If the two are not equivalent, the fitness program engine optionally modifies the subsequent workout schedule to compensate for the discrepancy.

The generated workout program is provided to the participant. Optionally, the method and system interact with a calendar or scheduling application that populate the participant's calendar and provide alerts and prompts to complete a workout.

In some embodiments, the fitness program provided to the participant at the start of the fitness program is a fluid fitness program that is modified based on participant performance and/or satisfaction.

Optionally, the fitness program engine includes one/or more optimization algorithms that modifies the fitness program based on performance. In some embodiments, the fitness program engine will recommend one or more modifications to the fitness program be made. In some embodiments, modifications will be made automatically and optionally, updates to the participant's calendar are sent.

Modifications include replacement of workout types, changes to specific workouts and replacement of workouts with rest days.

If the participant makes the modification to the workout, the system optionally sends a query to the participant with respect to the modification. For example, if the modification was an increase in weight used in a deadlift, the query relates to the perceived effort and optionally be a sliding scale.

In some embodiments, the fitness program engine is optionally configured to receive updates and modify the generated fitness program in response to the updates. In one embodiment, the system and method are configured to receive updates from fitness trackers which indicate that a particular workout has been completed or missed and update the fitness program accordingly. In some embodiments, updates to the fitness program include adding or subtracting segments or subsegments based on either tracked or inputted data.

In certain embodiments, the fitness program engine automatically substitutes a specific workout for another workout based on recent performance.

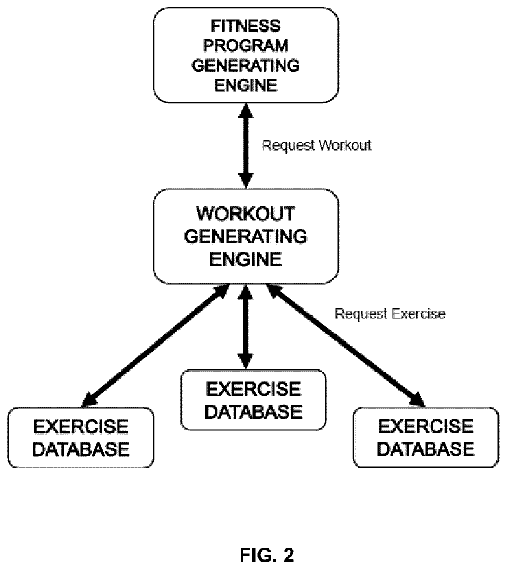

FIG. 2 shows a diagram for a computer implemented method for generating a workout. The method can be completed by the individual participating in the fitness/health program (“participant”), by a health care professional or a fitness professional or by initiated in response to a request for workout from the fitness program engine.

The method initiates with a step of receiving input via the interface regarding the participant that form one or more criteria that the workout must meet. Workout criteria include one or more workout type, workout length, required equipment amongst others. Workout type includes cardiovascular, strength training, mobility training, relaxation, stretching, weight-lifting, interval training, resistance training, high-intensity interval training, cross fit or combination.

Additional workout criteria include body part targeted including full body, core, legs, bum or “booty”, upper body, lower body, amongst others. Sports specific workouts may also be provided as a workout criteria. Sport specific workouts include exercises that are designed to enhance performance in a specific sport.

The workout generating engine based on the criteria in the requests selects or generates an appropriate workout template. Workout templates may be generic or workout type specific. Workout type-specific templates comprises cardiovascular workout template, strength training template, mobility training template, relaxation template, stretching template, weight-lifting template, interval training template, resistance training template, high-intensity interval training template, cross fit template or combination template. Optionally, workout type-specific templates are modular and the workout generating engine may be configured to combine two or more workout type-specific templates.

The workout generating engine based on the criteria will search the one or more exercise databases for exercises that meet the criteria and populate the selected workout template to generate a workout based on the participant's criteria.

In some embodiments, the workout generating engine queries the participant's profile and workout logs and modifies the workout based on past performance. Modifications include resistance, incline, weight, repetitions, and/or speed. Optionally, the workout generating engine will include one/or more optimization algorithms that modifies the workout based on performance. In some embodiments, the workout generating engine will recommend a modification to a specific workout be made.

### Example 1

The Present Example Illustrates a Method of Developing a Customized Workout Program Based on Personal Goals and/or Ability of an Embodiment of the Present Invention.

An advantage of this embodiment is the participant is given a customized schedule according to their personal goals, that allows the participant to be coached as if it were a 1/1 regimen. In specific embodiments of this method, group fitness classes are used with specific and distinct stimulus targets throughout the week, which allows for the creation individualized schedules. This is of value to the customer because of the substantial cost associated with 1/1 training. In this illustrated embodiment, participants get their own program, 2-way interactive trainer coached workouts+group environment motivation for much less, with the added convenience of accessing their workouts from anywhere, anytime.

A non-limiting example of a program layout is detailed below.

### Determining Ability Level

In certain embodiments, the participant's ability level is determined in order to develop a customized fitness program. Exemplary questions and answers are detailed below:

Question: Help us better personalize your customized fitness program by giving us information about your current comfort with fitness & ability level.
1. I have worked with a trainer for 2+years or a facility that educates on movement. I have plenty of experience with resistance training and high intensity interval training. I am comfortable with training however much it takes to achieve my goals (allowing me to sub in my sports). I am ready to go all in with 6+workouts weekly. (advanced)
2. I have worked with a trainer before to learn the fundamentals of movement and feel proficient. I am comfortable with a 5-6 day per week training routine (allowing me to sub in my sports). (intermediate 2)
3. I have trained with a trainer, facility or program that has given me the tools to understand the fundamentals of movement. The majority of my training has been guided (classes/instructed). I feel comfortable with working out 4-5× weekly. (intermediate 1)
4. I am relatively new to training but have some comfort with resistance training. I feel comfortable starting with 4× weekly training sessions to help me improve. (beginner 2)
5. I am brand new to working out. I don't have much of a background with resistance training. I am comfortable with 3-4× weekly training. (beginner 1)

The participant's activities or sports may be incorporated into the fitness program. In certain embodiments, an activity or sport that meets a predetermined intensity criteria can substitute a program workout.

In certain embodiments, the following activities do not meet the intensity criteria and will not qualify as a substitution for your workouts:
Walking
Playing with kids
Golfing with cart
Softball
Paddle boarding
Video games
Canoeing
Downhill skiing
Leisurely biking
Rollerblading

In certain embodiments, the following activities are intense enough to substitute into your schedule instead of a program workout:
Running more than 5 km
Soccer
Baseball
Hockey
Football
White water rafting
Golfing 18 holes without cart
Cross country skiing
Hiking uphill
Competitive/Timed Cycling

The fitness program may be further modified in light of the participant's goals. In certain embodiments, the program is modified after a predetermined length of time training with a program. In specific embodiments, the participant has trained with the program for at least two months before modifying. Non-limiting exemplary goals include better health, fat loss, muscle building, upper body building, lower body muscle building, and mobility/movement improvement. Exemplary workout schedules are detailed below and the exemplary workouts are defined as follows:

Rest refers to a rest day.

Activity refers to an activity outside of the workout plan.

Strong as used herein in reference to a particular workout refers to a 45 min full body, group training, balanced (anterior and posterior) pairings of upper and lower body movements that are sequenced to prioritized to a maximum localized sympathetic reaction of the target muscle groupings. The sequenced movements use balance, time and muscle failure as a constraint method to achieve muscle taxation to specific sites (the physiological response resulting in=hypertrophy).

Hardcore as used herein in reference to a particular workout refers to a 45 min full body, group training, balanced (upper & lower, anterior & posterior+athletic movement patterns) workout circuit designed to improve the participant's ability to perform repeat bouts of maximal effort. Training effect targeting the maximum caloric output using time and two intervals as the constraint to reaching maximum failure. 1 min of repeating 6-8 repetitions of performing the movement as hard as you can is followed by 2 max output bodyweight intervals.

Move as used herein in reference to a particular workout refers to a 45 min, full body, group training, balanced (anterior & posterior, internal and external rotation+core and dynamic movements) workout with a purpose of improving movement patterns and stimulating recovery. A sustained aerobic output stimulus is provided through a circuit of exercisers, whereas the sequence of movements are designed to prepare the participant for 2 of the dynamic exercises and 2 cardio demanding components of the workout. These movements that are used in preparation use integrated spinal stabilization as a foundation to prepare the client in different ways to perform dynamic movements more efficiently. This is achieved through using time and quality execution as the constraints to achieve improving movement quality.

Abs & Hiit as used herein in reference to a particular workout refers to a sequence of exercises, group training, 30 min workout that pair intervals (maximal intensity) with localized site taxation using time and failure as a constraint to create maximal sympathetic reaction.

Restore as used herein in reference to a particular workout refers to a 45 minute workout where Clients are put through a multitude of movements designed to focus on effective/quality movement patterns, while being encouraged to solely breathe through the nose for the duration of the workout.

UPPER BODY Sculpt as used herein in reference to a particular workout refers to a 30 min upper body, group training, hypertrophy focused class. Upper body muscle groups targeted (that vary each day performed) that create a maximal localized sympathetic reaction in the muscle-using timed work and muscle failure as the primary constraints to achieving hypertrophy. A sequence of 4 movements (1 compound, 1 shortened state dominant and 1 lengthened state) are used in sequence to create targeted muscle failure for [DAY 1—BACK & BICEPS, DAY 2—CHEST & TRICEPS, DAY 3—SHOULDERS+BICEPS+TRICEPS]

BOOTY builder as used herein in reference to a particular workout refers 30 min, group training, Glute/lower body hypertrophy focused class. Glute/Lower body muscle groups targeted (that vary each day performed) that create a maximal localized sympathetic reaction in the muscle-using timed work and muscle failure as the primary constraints to achieving hypertrophy. A sequence of 4 movements (1 compound, 1 shortened state dominant, 1 lengthened state & 1 Glute burnout) are used in sequence to create targeted muscle failure for [DAY 1-MULTI-FLEXION GLUTE (ankle, knee & hip flexing to tax the Glute), DAY 2-HINGE DOMINANT GLUTE (taxing the glutes primarily through hinge patterns), DAY 3-HIP THRUST DOMINANT GLUTE (taxing the glutes through hip thrust alternatives).

MOBILITY as used herein in reference to a particular workout refers group training circuit prioritizing the exploration of complete joint capacity. A 45 min sequence where movements are put together targeting the testing, expanding exploration of new range of motion are sequenced to improve the health and capacity of joint articulation through all (primarily end range) range of motion.
a. Better Health/Gradual Progression Schedule-A client would be recommended this option when they are content with their current routine and results. They would be looking to continue on this trajectory of success and be administered the below well rounded workout schedule based on their assessed ability level.
   i. Advanced:
      1. Monday-Strong
      2. Tuesday—Move/Activity
      3. Wednesday—Hardcore/Activity
      4. Thursday—Strong
      5. Friday—Move/Activity
      6. Saturday—Hardcore
      7. Sunday—Restore/Rest/Activity
   ii. Intermediate 2
      1. Monday—Strong
      2. Tuesday—Move/Activity
      3. Wednesday—Rest/Activity
      4. Thursday—Strong
      5. Friday—Move
      6. Saturday—Hardcore/Activity
      7. Sunday—Rest/Activity
   iii. Intermediate
      1. Monday—Strong
      2. Tuesday—Move/Activity
      3. Wednesday—Rest/Activity
      4. Thursday—Strong
      5. Friday—Move
      6. Saturday—Rest/Activity
      7. Sunday—Rest/Activity
   iv. Beginner 2
      1. Monday—Strong
      2. Tuesday—Move/Activity
      3. Wednesday—Rest/Activity
      4. Thursday—Strong
      5. Friday—Move
      6. Saturday—Rest/Activity
      7. Sunday—Rest/Activity
   v. Beginner 1
      3. Wednesday—Rest/Activity
      4. Thursday—Strong
      5. Friday—Rest/Activity
      6. Saturday—Rest/Activity
      7. Sunday—Restore or Move
b. Fat Loss Schedule-A client would be recommended this training schedule when they are interested in making fat loss their primary goal. It is not recommended to stay on this program for more than 2 months. Once the 2month segment is complete, it is recommended to change programs.
   i. Advanced:
      1. Monday—Strong
      2. Tuesday—Abs & Hiit/Activity
      3. Wednesday—Hardcore
      4. Thursday—Strong
      5. Friday—Abs & Hiit/Activity
      6. Saturday—Hardcore
      7. Sunday—Move/Activity
   ii. Intermediate 2:
      1. Monday—Strong
      2. Tuesday—Abs & Hiit
      3. Wednesday—Hardcore/Activity
      4. Thursday—Strong
      5. Friday—Abs & Hiit/Activity
      6. Saturday—Hardcore/Activity
      7. Sunday—Rest/Activity
   iii. Intermediate:
      1. Monday—Strong
      2. Tuesday—Abs & Hiit/Activity
      3. Wednesday—Hardcore
      4. Thursday—Strong
      3. Wednesday—Rest/Aclivily
      4. Thursday—Strong
      5. Friday—Move
      6. Saturday—Rest/Activity
      7. Sunday—Move/Activity
   v. Beginner
      1. Monday—Strong
      2. Tuesday—Move
      3. Wednesday—Rest/Activity
      4. Thursday—Strong
      5. Friday—Move
      6. Saturday—Rest/Activity
      7. Sunday—Rest/Activity
c. Muscle Building Schedule-A client would be recommended this training schedule when they are interested in making lean muscle growth their primary goals for body recomposition.
   i. Advanced:
      1. Monday—Strong
      2. Tuesday—Upper Body Sculpt
      3. Wednesday—Booty Builder/Activity
      4. Thursday—Strong
      5. Friday—Upper Body Sculpt replay (thursday)/Activity
      6. Saturday—Hardcore
      7. Sunday—Restore/Activity
   ii. Intermediate 2:
      1. Monday—Strong
      2. Tuesday—Upper Body Sculpt
      3. Wednesday—Booty Builder/Activity
      4. Thursday—Strong
      5. Friday—Booty Builder
      6. Saturday—Rest/Activity
      7. Sunday—Rest/Activity
   iii. Intermediate:
      1. Monday—Strong
      2. Tuesday—Upper Body Sculpt
      3. Wednesday—Rest/Activity
      4. Thursday—Strong
      5. Friday—Booty Builder
      6. Saturday—Rest/Activity
      7. Sunday—Rest/Activity
   iv. Beginner2:
      1. Monday—Strong
      2. Tuesday—Upper Body Sculpt/Activity
      3. Wednesday—Rest
      4. Thursday—Strong
      5. Friday—Booty Builder
      6. Saturday—Rest/Activity
      7. Sunday—Rest/Activity
   v. Beginner:
      1. Monday—Strong
      2. Tuesday—Mobility/Activity
      3. Wednesday—Rest
      4. Thursday—Strong
      5. Friday—Booty Builder
      6. Saturday—Rest/Activity
      7. Sunday—Rest/Activity
d. Upper Body Building Schedule-A client would be recommended this training schedule when they are interested in achieving lean muscle growth, primarily in their upper body muscle groups.
   i. Advanced
      1. Monday—Strong/Activity
      2. Tuesday—Upper Body Sculpt
      3. Wednesday—Upper Body Sculpt
      4. Thursday—Strong/Activity
      5. Friday—Upper Body Sculpt replay
      6. Saturday—Hardcore
      7. Sunday—Rest/Activity
   ii. Intermediate 2:
      1. Monday—Strong/Activity
      2. Tuesday—Upper Body Sculpt
      3. Wednesday—Upper Body Sculpt
      4. Thursday—Strong/Activity
      5. Friday—Upper Body Sculpt replay (thurs)
      6. Saturday—Rest/Activity
      7. Sunday—Rest/Activity
   iii. Intermediate:
      1. Monday—Strong/Activity
      2. Tuesday—Upper Body Sculpt
      3. Wednesday—Upper Body Sculpt
      4. Thursday—Strong/Activity
      5. Friday—Upper Body Sculpt replay (thurs)
      6. Saturday—Rest/Activity
      7. Sunday—Rest/Activity
   iv. Beginner:
      1. Monday—Strong/Activity
      2. Tuesday—Upper Body Sculpt
      3. Wednesday—Upper Body Sculpt
      4. Thursday—Upper Body Sculpt
      5. Friday—Rest/Activity
      6. Saturday—Rest/Activity
      7. Sunday—Rest/Activity
   v. Beginner:
      1. Monday-Rest/Activity
      2. Tuesday—Upper Body Sculpt
      3. Wednesday—Upper Body Sculpt
      4. Thursday—Upper Body Sculpt
      5. Friday—Rest/Activity
      6. Saturday—Rest/Activity
      7. Sunday—Rest/Activity
e. Lower Body lean muscle building schedule-A client would be recommended this training schedule when they are interested in making growth primarily for their lower body muscle groupings.
   i. Advanced:
      1. Monday-Booty Builder
      2. Tuesday—Strong Replay (monday)/Activity
      3. Wednesday—Booty Builder
      4. Thursday—Strong/Activity
      5. Friday—Booty Builder
      6. Saturday—Hardcore/Activity
      7. Sunday—Rest/Activity
   ii. Intermediate 2:
      1. Monday—Strong/Activity
      2. Tuesday—Booty Builder Replay (Mon)
      3. Wednesday—Booty Builder
      4. Thursday—Strong/Activity
      5. Friday—Booty Builder
      6. Saturday—Rest/Activity
      7. Sunday—Rest/Activity
   iii. Intermediate:
      1. Monday-Booty Builder
      2. Tuesday—Rest/Activity
      3. Wednesday—Booty Builder
      4. Thursday—Strong/Activity
      5. Friday—Booty Builder
      6. Saturday—Rest/Activity
      7. Sunday—Rest/Activity
   iv. Beginner 2:
      1. Monday-Booty Builder
      2. Tuesday—Rest/Activity
      3. Wednesday—Booty Builder
      4. Thursday—Strong/Activity
      5. Friday—Booty Builder
      6. Saturday—Rest/Activity
      7. Sunday—Rest/Activity
   v. Beginner 2:
      1. Monday-Booty Builder
      2. Tuesday—Rest/Activity
      3. Wednesday—Booty Builder
      4. Thursday—Rest/Activity
      5. Friday—Booty Builder
      6. Saturday—Rest/Activity
      7. Sunday—Rest/Activity
f. Mobility/Movement Improvement Schedule-A client would be recommended this training schedule when they are interested in improving their joint health & movement quality as their primary goal.
   i. Plan
      ii. Monday-Foundations
      iii. Tuesday—Mobility+Move
      iv. Wednesday—Foundations
      V. Thursday—Mobility+Strong
      vi. Friday—Foundations+Move if possible
      vii. Saturday—Rest
      viii. Sunday—Restore

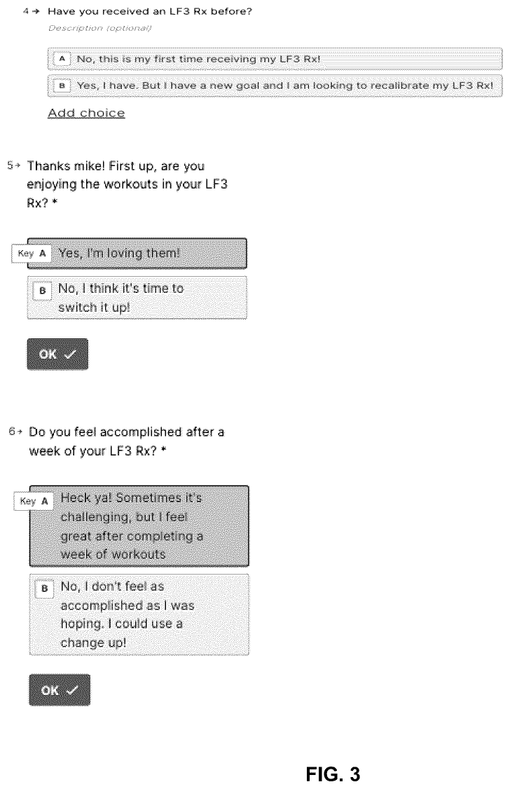

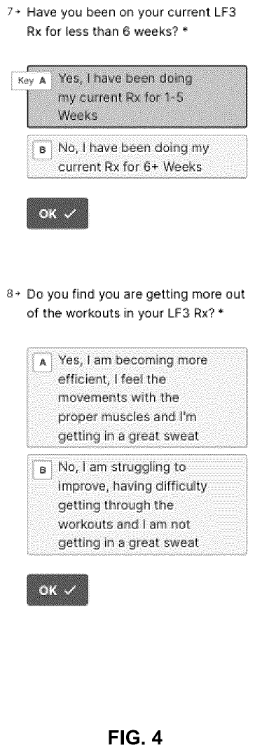

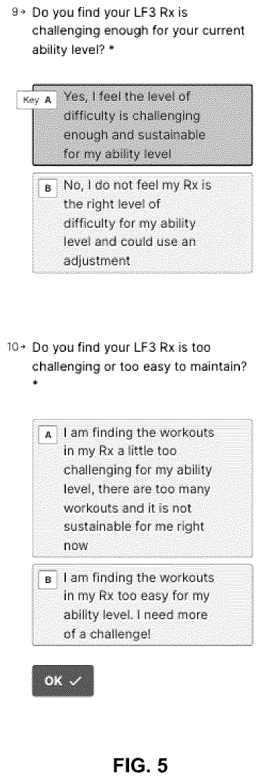

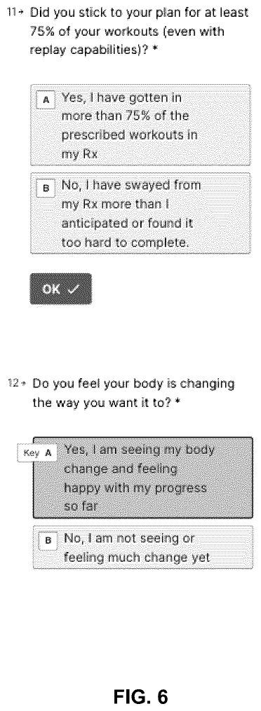

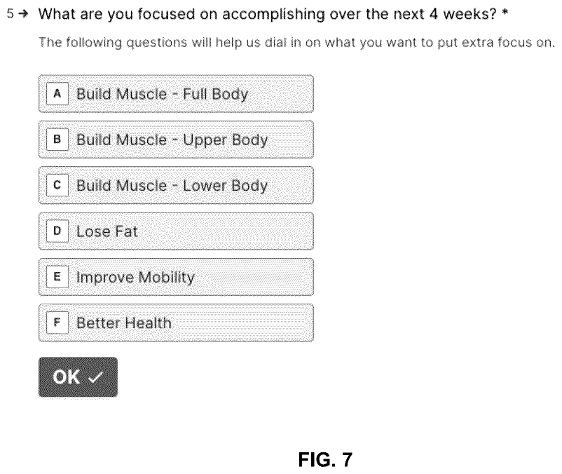

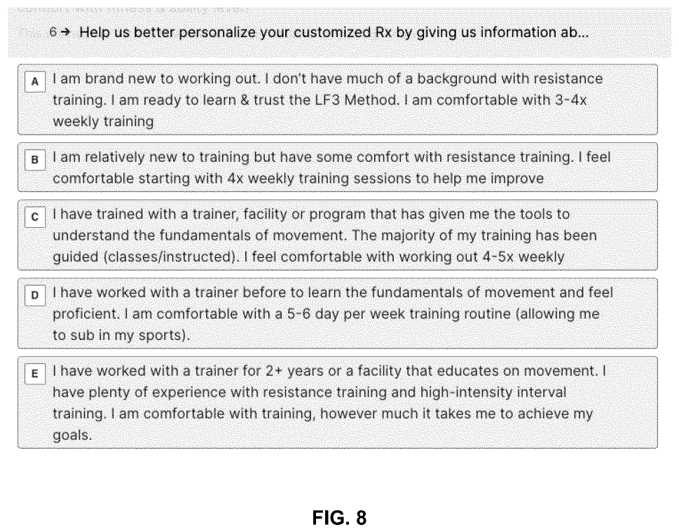

In certain embodiments, the program is used as follows:
1. Stage 1-Entry to the program: The first step in any clients's journey is to meet with one of the fitness professional to determine what is the most appropriate training schedule for the client—
   a. Should anyone have any injuries or potential hindrance to training, they will be prompted to book a 1/1 call with a specialist to customize their experience to accommodate any restrictions they may have.
   b. All those who are beginning their fitness journey for the first time OR for the first time in over 1 year, are recommended to start with the kickstart program
      i. The kickstart program is a 6-week program designed to teach the fundamental principles of training & get your nutrition in check. The program will get you off the couch and into a better mental and physical headspace.
      ii. Upon registration for the kickstart program, the client will meet with a specialist to set their schedule and receive their customized meal plan. The meal plan will be customized to the participant's goals (fat loss, muscle gain or maintenance).
   c. In the event that the client has a greater capacity/experience in working out, they will be sent a form so we can determine what their starting schedule should be. The client will be asked questions to determine workout ability level. Based on our determination, they will be given a beginner, intermediate or advanced level schedule to begin.
2. Stage 1 Alternative-Automation—
   a. In the event the client is unable to come into a physical location or interested in being self-sufficient, they have the ability to complete the automated platform. The platform provides the client with a custom schedule based on their needs input into the questionnaire.
   b. After identifying the client, the platform must determine if the client has completed this process before or if it is appropriate to get a new Rx. This is in place in order to improve adherence. It is important to identify if the client should be changing their program or if it is a premature decision. The series of questions are designed to take into account how the client is currently feeling about their training routine. The platform's purpose is to get the client to a physical and mental place where they are feeling accomplished. In the event there is a dissatisfaction or lack of adherence to the current program, they must be provided with a new one. This allows the client to take control of their choice in the journey, thus improving likelihood of sticking with the program.
   c. The following series is asked to identify the situation as shown in FIGS. 3 to 6.
   d. Once it has been determined if the client is eligible for receiving a new program, they are encouraged to proceed with the questionnaire.
   e. Step 1-Determine the client's fitness goals (FIG. 7). The client must give direction for the automation to provide scheduled training options. All programs are for a minimum of 4 weeks (long enough to see a specific adaptation occur).
   f. Step 2-Identify the client's exercise ability level (FIG. 8). The following questions are asked to determine the client's proficiency level for resistance, HIIT & cardiovascular training. There are a total of 5 ability level options which classify each LF3 Client. There is also an element of frequency of training introduced to the client at this stage. We are trying to set proper expectations for the individuals embarking on their fitness journey with our team.
   g. Step 3-Receipt of their program. Once the client has their program, they are equipped with knowing exactly what classes they should attend in order to accomplish the goals they have listed. Note—This entire process is client driven. The client chooses the goals they want to achieve. The client self reflected on their own ability level. Furthermore the client selected the most appropriate program for THEM all on their own. The adherence to this is extremely high due to the client driven approach.

In certain embodiments, the platform tracks, monitors and make changes to the individual's training schedule. In specific embodiments, the platform provides over 40 health metrics that are useful data points for the client to monitor throughout their journey with the program. The specific measurables that the platform uses to make changes to the individual schedules are as follows:
1. Total Fat Mass—This is the metric that provides insight as to whether or not your body fat is dropping. If that is the client's goal, their program is working when this number trends down.
2. Skeletal Muscle mass—This is the metric that provides insight into Muscle. The most important variable in determining/improving the rate at which someone can burn fat. If the client's goal is to improve muscle or lose fat, this number should trend upwards to show success of the program.
3. Total body water—This number determines if the client is in an appropriate state to scan or not. If this number is not within 5 lbs of the previous scan, the client must disregard the data points from the current scan and try to recreate a better state for their next one. Having an inflated or deflated body hydration will cause inflated or deflated muscle, in turn, also fat.

Once the client has starting point data, they can track to see if their program is making them progress towards their goals OR if they need to make an adjustment to their plan.

Many clients have body composition driven goals. This means they want to increase their muscle and decrease their fat. The combination of the two results in the change of an individual's shape. Strength can also be measured to a degree by evaluating muscle volume. In general, those with higher muscle mass have a greater strength capacity, to a certain point.

In certain embodiments, programs can be recalibrated manually with a fitness manager or automated through the platform. The platform can identify if the client is still making progress towards their goals or not. Simply-Should you change your workout schedule yes or no?

In certain embodiments, the platform takes 1. Hydration 2. Skeletal Muscle, 3. Fat Mass, into consideration when making this decision. The sequence to determine if the client gets a new training schedule or remains on the same is as follows, this is all based on the client GOALS:
1. Hydration—The platform compares the current hydration status to the most recent scan. IF the hydration status is above or below the previous by 5 lbs, it will list an error for the client. “This scan is not similar enough to your previous, please try again and recreate a more similar state”—It will recommend no coffee, no caffeine, no food and no workouts within 3 hours of your scan, in order to get a more accurate reading. If the client is within the 5 point window, the scan will count and be calculated to determine if the current plan is working or a new one is required.
2. Skeletal Muscle—The platform will compare the skeletal muscle to the skeletal muscle of the previous scan result. When the client's goal is to INCREASE muscle of upper, lower or full body. It will determine if this is higher or lower than the most recent scan. If it is lower, it will recommend the client completes another program in order to maximize the amount of muscle the client can attain. If the muscle is higher than the most recent scan, it will recommend that the client remain on the current program as the program is functioning.
3. Fat Mass—The platform will compare the current scan total fat mass reading to the most recent scan results for fat mass. When the clients goals are to DECREASE fat of upper, lower or full-body, the platform can make the most appropriate recommendation for workout adjustments. If the current fat mass is lower than the previous scan and their goal is to decrease fat, the current plan is working. The platform will recommend they stick to this plan for another 4 weeks. If the current fat mass is higher than the previous reading, the platform will recommend seeking a new Rx to more appropriately target their goals.

### Use Case Scenarios:

1. Female, 21 yrs, 50 Body Fat, 50 lbs Skeletal Muscle, Hydration 115, Ability level=advanced—Wants to lose fat
   a. Rx recommendation—
      i. Monday—Strong
      ii. Tuesday—Abs & Hiit/Activity
      iii. Wednesday—Hardcore
      iv. Thursday—Strong
      v. Friday—Abs & Hiit/Activity
      vi. Saturday—Hardcore
      vii. Sunday—Move/Activity

Although the present disclosure has been described with reference to specific features and embodiments thereof, it is evident that various modifications and combinations can be made thereto without departing from the disclosure. The specification and drawings are, accordingly, to be regarded simply as an illustration of the disclosure as defined by the appended claims, and are contemplated to cover any and all modifications, variations, combinations or equivalents that fall within the scope of the present disclosure.

1. A computer implemented method for generating a workout or a fitness program comprising a plurality of workouts, wherein each of the workout comprises one or more exercises, the method comprising:
receiving, by a server computing device, a request for a workout or a fitness program, wherein each request comprises one or more criteria, wherein each of the one or more criteria is selected from one or more fitness goals, workout type and workout length, wherein each request comprises a user submitting a request via a user interface on a user device, the user device communicating the request(s) with the server computing device optionally via the internet;
generating, by the server computing device, a workout or fitness program based on the one or more criteria;
displaying the workout or fitness program to the user; and
modifying the workout or fitness program in response to feedback comprising information relating to changes in exerciser specific information, wherein the exerciser specific information comprises physiological data and/or activity data.

2. The method of claim 1, wherein the physiological data is selected from age, sex, height, body weight, body fat, body composition, heart rate, aspiration rate, blood oxygenation, blood glucose, hydration, caloric expenditure, and VO_{2 max}; and
wherein the activity data is selected from exercise weight/resistance data, exercise repetition data, exercise duration, body motion/acceleration data, step data, stride length data, stride cadence data, distance traversal data, and pace/speed data.

3. (canceled)

4. The method of claim 1, wherein the generating the workout comprises:
selecting, by the server computing device, exercises from one or more databases of exercises; and
arranging selected exercises into a workout based on a workout template, a pre-determined set of rules and/or machine learning, wherein the step of arranging the selected exercises comprises selecting the workout template from one or more workout templates based on the one or more criteria and populating the templates with the selected exercises.

5. (canceled)

6. The method of claim 1, wherein displaying the workout or fitness program to the user comprises displaying a listing of exercises or workouts in the fitness program and optionally with instructions and/or videos on how to perform each of the exercises.

7. The method of claim 1, wherein the feedback is provided by user input and/or one or more monitoring devices, optionally wherein the feedback is provided automatically, wherein the one or more monitoring devices is a smart watch or fitness monitor.

8. The method of claim 1, wherein a subsequent workout in a fitness program or a subsequent exercise in a workout is automatically modified in response to the feedback.

9. (canceled)

10. A system for generating a workout having a plurality of exercises, the system comprising:
one or more exercise databases; and
a workout generating engine located on a server computing device operatively connectable to one or more user interfaces,
the workout generating engine comprising one or more workout templates stored in a memory; the workout generating engine configured to retrieve one or more exercises from the one or more exercise databases and populate a selected template to generate a workout based on user workout criteria,
wherein the workout generating engine is further configured to modify the generated workout in response to information relating to changes in exerciser specific information comprising physiological data and/or activity data.

11. The system for generating a workout of claim 10, wherein each entry in the one or more exercise database comprises exercise name and associated metadata, and optionally one or more of a video or http link to a video showing the exercise, one or more keywords, one or more diagrams showing the exercise and written description.

12. The system for generating a workout of claim 10, wherein the one or more workout templates comprises one or more workout type—specific templates, wherein the one or more workout type-specific templates comprises cardiovascular workout template, strength training template, mobility training template, relaxation template, stretching template, weight-lifting template, interval training template, resistance training template, high-intensity interval training template, cross fit template or combination template,
wherein the workout type-specific templates are modular, and/or
wherein the workout generating engine is configured to combine two or more workout type-specific templates.

13-14. (canceled)

15. The system for generating a workout of claim 10, further comprising saving the generated workout in a workout database.

16. The system for generating a workout of claim 10, wherein the user interface is configured to allow for selection of exerciser and wherein the workout generating engine is configured to retrieve one or more exerciser profiles.

17. The system for generating a workout of claim 10, wherein the physiological data is selected from age, sex, height, body weight, body fat, body composition, heart rate, aspiration rate, blood oxygenation, blood glucose, hydration, caloric expenditure, and VO_{2 max}; and
wherein the activity data is selected from exercise weight/resistance data, exercise repetition data, exercise duration, body motion/acceleration data, step data, stride length data, stride cadence data, distance traversal data, and pace/speed data.

18. (canceled)

19. The system for generating a workout of claim 10, wherein the system is configured to receive third-party data, wherein the third-party data is wearable fitness device data or health data, and wherein the system is optionally configured to output the generated workout on a display of a personal computing device.

20-21. (canceled)

22. A system for generating a fitness program having a plurality of workouts, the system comprising:
a fitness program generating engine located on a server computing device operatively connectable to one or more user interfaces,
the fitness program generating engine comprising one or more fitness program templates stored in a memory; the fitness generating engine configured to retrieve one or more workouts from the system for generating a workout as defined in claim 10 and populate a selected template to generate a fitness program based on user criteria.

23. A computer implemented method for generating a fitness program for a participant comprising a plurality of workouts, wherein each of the workout comprises one or more exercises, the method comprising:
receiving, by a server computing device, a request for a fitness program, wherein each request comprises one or more criteria, wherein each of the one or more criteria is selected from fitness level of participant, one or more fitness goals of the participant, current activities of the participant, wherein each request comprises a user submitting a request via a user interface on a user device, the user device communicating the request(s) with the server computing device optionally via the internet;
generating, by the server computing device, a workout or fitness program based on the one or more criteria;
displaying the workout or fitness program to the user,
modifying the workout or fitness program in response to feedback comprising information relating to changes in exerciser specific information, wherein the exerciser specific information comprises physiological data and/or activity data.

24. The method of claim 23, wherein the physiological data is selected from age, sex, height, body weight, body fat, body composition, heart rate, aspiration rate, blood oxygenation, blood glucose, hydration, caloric expenditure, and VO_{2 max}; and
wherein the activity data is selected from exercise weight/resistance data, exercise repetition data, exercise duration, body motion/acceleration data, step data, stride length data, stride cadence data, distance traversal data, and pace/speed data.

25. (canceled)

26. The method of claim 23,
wherein the generating the fitness program comprises:
selecting, by the server computing device, exercises from one or more databases of exercises, based on the one or more criteria; and
arranging selected exercises into one or more workouts based on a fitness program template, a pre-determined set of rules and/or machine learning.

27. The method of claim 1, wherein the workout or fitness program includes one or more of a group workout, an on-line workout, a workout with a personal trainer, and individual exercise, and
wherein the workout or fitness program optionally comprises a diet plan, the diet plan being customized based on the user's fitness and health status.

28. The method of claim 1, further comprising interactive communication with a trainer.

29. (canceled)

30. The system of claim 10, wherein the physiological data comprises bioimpedance analysis data and/or 3-D body scan data.
